(19) United States
(12) Patent Application Publication
(10) Pub. No.: US 2022/0280587 A1
(43) Pub. Date: Sep. 8, 2022

(54) MEDICAL GELS CONTAINING A QUATERNARY AMMONIUM COMPOUND, PHENOXYETHANOL, ALOE VERA JUICE, AND NATURAL SPORICIDAL MOISTURIZERS

(71) Applicant: Mark Edward Fenzl, Sarasota, FL (US)

(72) Inventor: Mark Edward Fenzl, Sarasota, FL (US)

(21) Appl. No.: 17/190,628

(22) Filed: Mar. 3, 2021

(51) Int. Cl. A61K 36/886; A61K 8/41; A61K 8/9794; A61K 8/34; A61Q 17/00; A01N 65/42; A61K 47/18; A61K 47/10

(52) U.S. Cl. CPC A61K 36/886; A61K 8/416; A61K 8/9794; A61K 8/347; A61Q 17/005; A01N 65/42; A61K 47/186; A61K 47/10

(57) ABSTRACT

A hand sanitizer that is a combination of benzalkonium chloride, phenoxyethanol, aloe vera juice, and natural sporicidal moisturizers. The hand sanitizer can also be used as a burn gel with full broad-spectrum bacterial coverage, full broad-spectrum bacterial spore coverage, full broad-spectrum fungal coverage, skin irritation protection, oxidizing protection, humidifying effects, and moisturizing effects.

### CROSS-REFERENCE TO RELATED APPLICATIONS

This application claims the benefit of U.S. Provisional Application No. 63/102,158, filed on Mar. 30, 2020. The entire disclosure of the above application is hereby incorporated herein by reference.

### FIELD

The present disclosure relates to hand sanitizers containing benzalkonium chloride, phenoxyethanol, aloe vera juice, and natural sporicidal preservatives. This unique product uses a combination of natural synergistic sporicidal preservatives that protect the skin to lower irritation and protect from infection. This hand sanitizer may be used as a combination hand sanitizer and burn gel.

### BACKGROUND

This section provides background information related to the present disclosure which is not necessarily prior art.

Hand sanitizers are used to protect people from bacterial, fungal, and viral infection. Contamination of hand sanitizers with microbes can lead to infection of a patient or healthcare provider. To protect patients and healthcare providers, hand sanitizers have preservatives that inhibit or kill microbes.

The majority of hand sanitizers contain ethanol or isopropyl ethanol at a concentration of 60%-70%. This is effective at killing bacteria and the majority of viruses, but dries the skin and makes it susceptible to cracks that can lead to infection.

There are known hand sanitizers that contain benzalkonium chloride as the preservative, but benzalkonium chloride alone is ineffective against many fungi and bacterial spores at skin contact concentrations. It also has an irritating effect on the skin at bactericidal concentrations, which can cause a break in the skin and make the skin susceptible to infection.

There are a variety of natural chemicals that can be used to protect hand sanitizers from microbial contamination and oxidation. Using natural preservatives reduces the risks of irritation, allergic reaction, and cancer that can occur with synthetic preservatives. People often prefer natural chemicals to synthetic preservatives for this reason. Providing hand sanitizers that are preserved with natural chemicals will provide preservative protection while reducing side effects and providing a product on the market that is reassuring to the public. A problem with natural chemicals is that plant products that are resistant to some microorganisms are susceptible spoilage by other microorganisms.

Aloe vera juice comes from the aloe vera plant. It is bactericidal of Pseudomonas aeruginosa at a an approximate concentration of 10% and inhibits Pseudomonas aeruginosa at an approximate concentration of 5%. It has a hydrating effect on the skin.

Sorbic acid is a found in rowan berries, inhibits bacteria that form spores, and has an antifungal effect. Sorbic acid lowers the pH and has synergistic effects with phenoxyethanol and vanillin.

Phenoxyethanol is a chemical found in green tea and chicory that inhibits bacteria and fungi. Phenoxyethanol has a synergistic antibacterial effect with potassium sorbate and sorbic acid.

Monolaurin is a monoglyceride found in breast milk and coconuts. It inhibits and kills bacterial bacteria, bacterial spores, fungi, and enveloped viruses. It has a synergistic antibacterial effect with a variety of acids and a stronger sporicidal effect at lower pH.

Vanillin is a aldehyde found in natural vanilla. It inhibits bacterial spores and fungi. It has synergistic antifungal effect with sorbic acid.

There is a need for ammonium quaternary compound hand sanitizers containing natural moisturizing antivirals that protect from dry skin, bacterial spores, oxidation, and skin irritation.

### SUMMARY

In concordance with the instant disclosure, a moisturing, non-irritating, alcohol-free sporicidal sanitizer gel has surprisingly been discovered.

The hand sanitizer may include a quaternary ammononium compound, pheonxyethanol, aloe vera juice, a monoglyceride, an aldehyde, and a sorbate preservative. A benefit of using a quaternary ammonium compound is that they can be used as a sanitizer. Not only will the quaternary ammonium compound protect the product from contamination, but it will also protect the patient from any microbes on the skin. A benefit of natural antimicrobials is that microorganisms are developing resistance to synthetic preservatives. Not only can natural antimicrobials protect the product from antibiotic resistant microbes, but by killing bacteria that are resistant to antibiotics prevents the spread of antibiotic resistant microbes. The natural preservative sorbic acid lowers the pH of the product that has a synergistic effect with monolaurin and has synergistic antimicrobial effects with the natural preservatives phenoxyethanol and vanillin.

There are other benefits of using natural chemicals as a part of a combination preservative. Plant extracts that have antimicrobial properties often have antioxidant properties. This protects the product from degradation when the product is exposed to ultraviolet light or free radicals. Combining low levels of synthetic chemicals with moisturizing antiviral plant extracts provides a synergistic effect of protecting the product from microbial contamination and protecting the synthetic preservative from degradation.

The present technology can be a gel that is able to protect the user from infection by killing a broad spectrum of bacteria, killing bacterial spores, killing fungi, moisturizing the skin, and providing antioxidant protection. Benzalkonium chloride is not used at higher concentrations due to skin irritation. Benzalkonium chloride does not kill bacterial spores. Monolaurin, sorbic acid, and vanillin inhibit and kill bacterial spores. Other variations can include a combination hand sanitizer and surgical sanitizer, ultrasound gel, surgical sanitizer, surgical sanitizer and wound gel, etc.

### DETAILED DESCRIPTION

The following description of technology is merely exemplary in nature of the subject matter, manufacture and use of one or more inventions, and is not intended to limit the scope, application, or uses of any specific invention claimed in this application or in such other applications as can be filed claiming priority to this application, or patents issuing therefrom. Regarding methods disclosed, the order of the steps presented is exemplary in nature, and thus, the order of the steps can be different in various embodiments, including where certain steps can be simultaneously performed. “A” and “an” as used herein indicate “at least one” of the item is present; a plurality of such items can be present, when possible. Except where otherwise expressly indicated, all numerical quantities in this description are to be understood as modified by the word “about” and all geometric and spatial descriptors are to be understood as modified by the word “substantially” in describing the broadest scope of the technology. “About” when applied to numerical values indicates that the calculation or the measurement allows some slight imprecision in the value (with some approach to exactness in the value; approximately or reasonably close to the value; nearly). If, for some reason, the imprecision provided by “about” and/or “substantially” is not otherwise understood in the art with this ordinary meaning, then “about” and/or “substantially” as used herein indicates at least variations that can arise from ordinary methods of measuring or using such parameters.

Although the open-ended term “comprising,” as a synonym of non-restrictive terms such as including, containing, or having, is used herein to describe and claim embodiments of the present technology, embodiments can alternatively be described using more limiting terms such as “consisting of” or “consisting essentially of” Thus, for any given embodiment reciting materials, components, or process steps, the present technology also specifically includes embodiments consisting of, or consisting essentially of, such materials, components, or process steps excluding additional materials, components or processes (for consisting of) and excluding additional materials, components or processes affecting the significant properties of the embodiment (for consisting essentially of), even though such additional materials, components or processes are not explicitly recited in this application. For example, recitation of a composition or process reciting elements A, B and C specifically envisions embodiments consisting of, and consisting essentially of, A, B and C, excluding an element D that can be recited in the art, even though element D is not explicitly described as being excluded herein.

Disclosures of ranges are, unless specified otherwise, inclusive of endpoints and include all distinct values and further divided ranges within the entire range. Thus, for example, a range of “from A to B” or “from about A to about B” is inclusive of A and of B. Disclosure of values and ranges of values for specific parameters (such as amounts, weight percentages, etc.) are not exclusive of other values and ranges of values useful herein. It is envisioned that two or more specific exemplified values for a given parameter can define endpoints for a range of values that can be claimed for the parameter. For example, if Parameter X is exemplified herein to have value A and also exemplified to have value Z, it is envisioned that Parameter X can have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if Parameter X is exemplified herein to have values in the range of 1-10, or 2-9, or 3-8, it is also envisioned that Parameter X can have other ranges of values including 1-9, 1-8, 1-3, 1-2, 2-10, 2-8, 2-3, 3-10, 3-9, and so on.

The term “ultrasound gel” refers to scanning gel or gels, transmission gel or gels, ultrasound gel or gels, ultrasound lotion or lotions, or any other solution designed or sold for the purpose of transmitting ultrasound waves.

The term “hand sanitizer” refers to gels used to sanitize the skin.

The present disclosure relates to a medical gel including quaternary ammonium compounds and natural moisturizing sporocidal antivirals. The medical gel can be a hand sanitizer, a surgical sanitizer, an ultrasound gel, a massage gel, a burn gel, and a wound gel, as non-limiting examples.

The medical gel or sanitizer can include a quaternary ammonium compound. Quaternary ammonium compounds (QACs) can be used as disinfectants. There are numerous commercially available products and formulations containing quaternary ammonium compounds. Quaternary ammonium compounds are cationic surfactants (positively charged surface-active agents) that impact cell walls and membranes after relatively long contact times. Quaternary ammonium compounds have a permanent positive charge, which can allow them bind to a negatively charged surface of most microbes.

QACs can be used at concentrations ranging from about 200 to 400 ppm for a hard contact surfaces, as a non-limiting example. QACs are generally very stable, mostly unaffected by pH levels, and remain effective on a contact surface for a long time. An example of a QAC is benzalkonium chloride, which is often used as a cleaner and sanitizer for various food surfaces, both at home and in industrial applications such as dairy equipment. As one non-limiting example, the QAC can be benzalkonium chloride.

Pheonxyethanol is a natural chemical that inhibits bacteria and fungi. It has a synergistic antimicrobial effect with sorbic acid.

Various types of plant extracts can be included in the medical gel. As a non-limiting example, the plant extract can be aloe vera juice. Aloe vera juice has been shown to inhibit viruses. Aloe vera juice is also bactericidal of Pseudomonas aeruginosa. Aloe vera juice can be combined with benzalkonium chloride in the medical gel. Aloe vera juice can replace a portion of the benzalkonium chloride in the medical product. For example, benzalkonium chloride kills Pseudomonas aeruginosa at a concentration of 0.13%, a level that causes skin irritation. But the concentration of benzalkonium chloride can be lowered to 0.10% when aloe vera juice is present at 10%. Advantageously, the lower concentration of benzalkonium chloride at 0.10% is non-irritating. Aloe vera juice is also a hydrating agent.

Various types of fatty acids can be included in the medical gel. The medical gel can also have a fatty acid, a fatty acid salt, a fatty acid ester. Monoalurin is a natural monoglyceride found in breast milk and coconut oil. It inhibits bacteria, bacterial spores, fungi, and enveloped viruses. It moisturizes and protects the skin and has a synergistic antibacterial effect when combined with acids and at lower pH.

Various types of aldehydes can be included in the medical gel. A non-limiting example of an aldehyde is vanillin. Vanillin is a natural plant chemical that kill bacteria, bacterial spores, and fungi. It also has antioxidant properties. Vanillin has a synergistic antifungal effect with sorbic acid. Demonstration of the sporicidal effects of vanillin can be found in Table 3.

Various plant acids can be included including sorbic and benzoic preservatives that are preservatives that are found in plants. Potassium sorbate, that is more water soluble, converts to sorbic acid for its preservative effect.

Sorbitol has been shown to inhibit some viruses by binding to binding sites, thereby preventing the virus from binding to cells. Sorbitol can also desirably act as a humectant. This protects the skin from drying.

Various components of the medical gel can act in concert. While bound to no particular theory, it is believed that vanillin and sorbic acid can work synergistically to improve the antifungal ability of the medical gel. For example, vanillin can be combined with potassium sorbate in the medical gel. The FIC (Fractional Inhibitory Concentration) is shown below in Table 1. The MIC of potassium sorbate with vanillin is seen in Table 2. Potassium sorbate is active when it naturally converts to sorbic acid.

One particular non-limiting example, the medical gel can be a hand sanitizer. The hand sanitizer can include benzalkonium chloride at a product concentration of 1,000 parts per million (0.1%), phenoxyethanol at a product concentration of 3,200 parts per million (0.32%), vanillin at a product concentration of 5,000 parts per million (0.5%), sorbic acid at a product concentration of 3,000 parts per million (0.3%), monolaurin at a product concentration of 10,000 parts per million (1%), hydroxypropyl methylcellulose at a product concentration of 10,000 parts per million (1%), sorbitol at a product concentration of 40,000 parts per million (4%), and aloe vera juice at quantities sufficient to reach 100%.

This non-limiting example of a medical gel can be used as a combination hand sanitizer and burn gel.

The medical gel can be formulated as various types of hand sanitizers. Hand sanitizers may include alcohols such as isopropyl alcohol, ethanol (ethyl alcohol), or n-propanol. Compounds such as glycerol may be added to prevent drying of the skin. Hand sanitizers can optionally contain fragrances.

The medical gel can be formulated as various types of surgical sanitizers. Surgical sanitizers can include alcohols. A chlorhexidine gluconate solution may also be used in the surgical sanitizer.

The medical gel can be formulated as various types of massage gel. Massage gels can contain oils, which may improve the use for massages. For example, essential oils can improve the scent of the medical gel.

The medical gel can be formulated as various types of burn gels. Burn gels can include an analgesic. For example, the analgesic can be lidocaine. The burn gel can also include additional vitamins, such as vitamin E.

The medical gel can be formulated as various types of wound gels. Wound gels can include an analgesic. For example, the analgesic can be lidocaine. The wound gel can also include a skin protectant, such as allantoin.

Additionally, as may be presented in the claims below, the language “consisting of” is intended to exclude any ingredient not specified in the claim. Accordingly, the medical gel consisting of certain ingredients described hereinabove includes only those ingredients.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments can be embodied in many different forms, and that neither should be construed to limit the scope of the disclosure. In some example embodiments, well-known processes, well-known device structures, and well-known technologies are not described in detail. Equivalent changes, modifications and variations of some embodiments, materials, compositions and methods can be made within the scope of the present technology, with substantially similar results.

TABLE 1
FIC isobolograms for combinations of potassium
sorbate and vanillin against P. digitatum (O),
P. glabrum (X), and P. italicum (+).

| FIC | | | | | | | | | | |
|---|---|---|---|---|---|---|---|---|---|---|
| Potassium | FIC Vanillin | | | | | | | | | |
| Sorbate | 0.1 | 0.2 | 0.3 | 0.4 | 0.5 | 0.6 | 0.7 | 0.8 | 0.9 | 1.0 |
| 0 | | | | | | | | | | |
| 0.1 | | | | | | | OX+ | | | |
| 0.2 | | | | | | | | | | |
| 0.3 | | | O | | + | | | | | |
| 0.4 | | | | X | | | | | | |
| 0.5 | | | | | | | | | | |
| 0.6 | | | | | | | | | | |
| 0.7 | | X | | | | | | | | |
| 0.8 | | | | | | | | | | |
| 0.9 | | | | | | | | | | |
| 1.0 | | | | | | | | | | |

TABLE 2

| | |
|---|---|
| Concentration | |
| Potassium | Vanillin |
| Sorbate PPM | MIC PPM |

P. digitatum

| | |
|---|---|
| 0 | 1,100 |
| 50 | 300 |
| 100 | 100 |
| 150 | 0 |

P. glabrum

| | |
|---|---|
| 0 | 1,200 |
| 100 | 1,100 |
| 300 | 500 |
| 500 | 100 |
| 700 | 0 |

P. italicum

| | |
|---|---|
| 0 | 1,100 |
| 100 | 300 |
| 150 | 100 |
| 200 | 0 |

TABLE 3
Inhibitory activity of aldehydes against
C. botulinum spores at 32 degrees in broth.

| | Sporostatic | Sporicidal |
|---|---|---|
| | MIC | MIC |
| Test Compound | (mM) | (mM) |
| Benzaldehyde | 0.60 | 625 |
| Acetaldehyde | 0.78 | >2500 |
| Cinnamaldehyde | 0.78 | >2500 |
| Piperonal | 0.60 | >2500 |
| Isobutyraldehyde | 0.60 | 39 |
| Phenylacetaldehyde | 0.60 | >2500 |
| α-Amylcinnamaldehyde | 0.60 | 2500 |
| Pyruvaldehyde | 2.44 | 156 |
| Vanillin | 0.60 | 1.25 |
| Phenylglyoxal | 0.60 | >2500 |
| Hepaldehyde | 0.60 | >2500 |
| Valeraldehyde | 2.44 | 156 |

What is claimed is:

1. A medical sanitizer, burn gel, wound gel, or ultrasound gel comprising:
aloe vera juice at a concentration sufficient to kill Pseudomonas aeruginosa, a quaternary ammonium compound at a concentration of 0.1% or less, and phenoxyethanol at a concentration less than 1%.

2. The product in claim 1, wherein the product is a hand sanitizer.

3. The product in claim 1, wherein the product is a surgical sanitizer.

4. The product in claim 1, wherein the product is an ultrasound gel.

5. The product in claim 1, wherein the product is a combination of at least two two from the group consisting of: hand sanitizer, surgical sanitizer, ultrasound gel, burn gel, and wound gel.

6. An medical sanitizer, ultrasound gel, or wound gel comprising
a sorbate, a monoglyceride, and an aldehyde.

7. The product in claim 6, wherein the product is a hand sanitizer.

8. The product in claim 6, wherein the product is a surgical sanitizer.

9. The product in claim 6, wherein the product is an ultrasound gel.

10. The product in claim 6, wherein the product is a combination of at least two two from the group consisting of: hand sanitizer, surgical sanitizer, ultrasound gel, massage gel, burn gel, and wound gel.

11. A medical sanitizer or ultrasound gel comprising
a juice as the largest component of the product by volume.

12. The product in claim 11, wherein the product is a hand sanitizer.

13. The product in claim 11, wherein the product is a surgical sanitizer.

14. The product in claim 11, wherein the product is an ultrasound gel.

15. The product in claim 11, wherein the product is a combination of at least two from the group consisting of: hand sanitizer, surgical sanitizer, ultrasound gel, massage gel, burn gel, and wound gel.
